(19) United States
(12) Patent Application Publication
(10) Pub. No.: US 2024/0294125 A1
(43) Pub. Date: Sep. 5, 2024

(54) SYSTEM FOR SANITIZING USER SPACE OF A VEHICLE AND METHODS THEREOF

(71) Applicant: Mahindra & Mahindra Limited, Chengalpattu, Tamilnadu (IN)

(72) Inventors: Shankar ANAND S, Chengalpattu (IN); Venugopal SHANKAR, Chengalpattu (IN); N. PRASANNA, Chengalpattu (IN)

(21) Appl. No.: 18/573,118

(22) PCT Filed: Jun. 22, 2022
(86) PCT No.: PCT/IN2022/050575

§ 371 Date: Dec. 21, 2023

(30) Foreign Application Priority Data
Jun. 22, 2021 (IN) 202141028051

(51) Int. Cl. B60R 15/00; A61L 2/22; A61L 2/24; B60N 2/60

(52) U.S. Cl. CPC B60R 15/00; A61L 2/22; A61L 2/24; B60N 2/60; A61L 2202/14; A61L 2202/15; A61L 2202/25

(57) ABSTRACT

Disclosure herein relate to a sanitization system comprising at least one sanitizer dispensing unit integrated in a seat cover for sanitizing user space in a vehicle. Further, disclosure herein relate to a method for sanitizing the user space of the vehicle. Furthermore, disclosure herein relate to a method for estimating degree of sanitization required in the user space. Disclosure herein relate to a method for estimating the degree of sanitization required in user space, wherein the system can effectively sanitize the user space in vehicle and intimating the hygiene status of vehicle before passenger starting the ride thereby protecting passengers from exposure to potentially harmful microbes accumulated on passenger seats as well other interior spaces in the vehicle.

### CROSS REFERENCE TO RELATED APPLICATION

This application is based on and derives the benefit of Indian Complete Application 202141028051 filed on 22 Jun. 2021, the contents of which are incorporated herein by reference.

### TECHNICAL FIELD

Embodiments disclosed herein relate to a sanitization system comprising at least one sanitizer dispensing unit integrated in a seat cover for sanitizing user space in a vehicle. Further, embodiments herein relate to a method for sanitizing the user space of the vehicle. Furthermore, embodiments herein relate to a method for estimating degree of sanitization required in the user space to accomplish effective sanitization of the user space in the vehicle.

### BACKGROUND

In order to provide an adequate level of hygiene and cleanliness in a shared mobility vehicle such as buses, ambulance, passenger vehicle and the like, the interior spaces of the vehicle are cleaned at regular intervals. For example, in case of pandemic or COVID-19, the passenger may hesitate to travel in such shared mobility vehicles due to fear of infection. An infected person can potentially spread the disease to many people while travelling in mass transit like buses and trains thereby these vehicles will also be less preferred due to fear of infection. Therefore, there is at least a demand to assure hygienic conditions in interior spaces of such vehicles.

Further, unfortunately, residue, germs, lice, bacteria and viruses originating from the previous user can be deposited onto the seat and as well other interior spaces in the vehicle. This can be easily transferred to the next user while sitting on the same seat. Because of this, passenger seats installed in the various modes of public transportation vehicles can be a breeding ground for virus and bacteria, which can cause colds, flu and other infectious diseases.

This common event can result in a significant amount of germs, bacteria and virus being, transferred onto the eyes, nose, mouth, neck, arms, and hands of the user thereby increasing the possibility of getting infected.

Therefore, there exists a need for a system that would protect passengers from exposure to potentially harmful microbes accumulated on passenger seats and interior space of a vehicle Further, there exists a need for a retrofit seat cover with integrated sanitizing system which is intended to provide such protection and also serve as a means of disinfecting the user space of the shared mobility vehicle after use or completing the ride in the shared mobility vehicle. Furthermore, there exists a need for the system which can perform the sanitization process in the vehicle effectively and provide intimation or alert to the passenger about the hygiene status before starting the ride in the vehicle.

### OBJECTS

The principal object of embodiments herein is to disclose a system for sanitizing a user space of a vehicle.

Another object of embodiments herein is to provide a method for sanitizing the user space of the vehicle.

Another object of embodiments herein is to provide the system for operating at least one sanitizer dispensing unit integrated in one of a seat cover, seat, bed cover, bed, door handle, roof, dashboard, steering wheel, and heating, ventilation and air conditioning (HVAC) module of the vehicle, where the sanitizer dispensing unit can effectively dispense the sanitizer fluid to user space of the vehicle.

Another object of embodiments herein is to provide a seat cover which is simple in construction and allows retrofitting of sanitizer fluid flow channels and sanitizer dispensing unit therein to effectively dispense the sanitizer fluid to user space of the vehicle.

Another object of embodiments herein is to disclose a system and method for estimating the degree of sanitization required in the user space of the vehicle in accordance to geographical location of the user(s).

Another object of embodiments herein is to allow the user of vehicle to select the level of sanitization required in user space before starting the ride.

Another object of embodiments herein is to disclose the seat cover with integrated sanitizer fluid flow channels and sanitizer dispensing unit to dispense the sanitized fluid to the user space of the vehicle.

Yet another object of embodiments herein is to disclose a method for operating at least one sanitizer dispensing unit in the vehicle.

Yet another object of embodiments herein is to provide the system for intimating/alerting the hygiene status of the vehicle before passenger starting the ride.

These and other aspects of the embodiments herein will be better appreciated and understood when considered in conjunction with the following description and the accompanying drawings. It should be understood, however, that the following descriptions, while indicating at least one embodiment and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF FIGURES

Embodiments herein are illustrated in the accompanying drawings, throughout which like reference letters indicate corresponding parts in the various figures. The embodiments herein will be better understood from the following description with reference to the drawings, in which:

FIG. 1 depicts a sanitization system in a vehicle, wherein the sanitization system comprises a seat cover, sanitizing dispensing units, a sanitizer circulating device and a controller unit, where the controller unit is in communication with a sensor module and an indication module, according to embodiments as disclosed herein;

FIG. 2 depicts the seat cover retrofitted onto a seat of the vehicle, according to embodiments as disclosed herein;

FIG. 3A and 3B depict the seat cover with module, wherein the module comprises the at least one sanitizing dispensing unit and sanitizer fluid flow channels, according to embodiments as disclosed herein;

FIG. 4 is a flowchart depicting a process of sanitizing the user space and regeneration of sanitizer fluid from the sanitized user space of the vehicle, according embodiments as disclosed herein; and

FIG. 5 is a flowchart depicting a process of estimating the degree of sanitization level required in user space of the vehicle, according to embodiments as disclosed herein.

### DETAILED DESCRIPTION

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

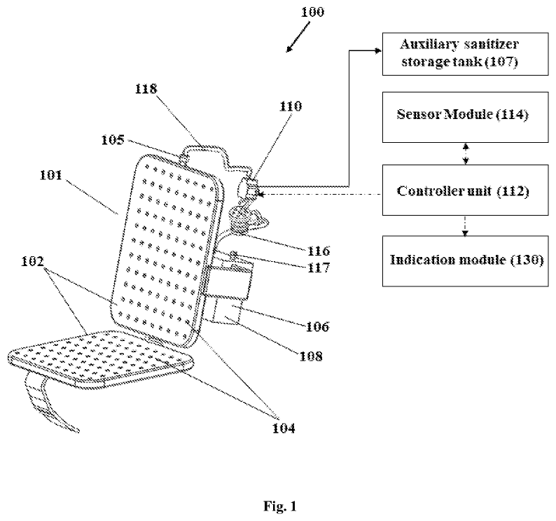

The embodiments herein achieve a system and a method for sanitizing a user space of the vehicle. Further, embodiments herein achieve a system for operating at least one sanitizer dispensing unit integrated in a seat cover of the vehicle, where the sanitizer dispensing unit effectively dispense the sanitizer fluid to user space of vehicle. Further, embodiments herein achieve a method and system for estimating the degree of sanitization required in user space of the vehicle in accordance to geographical location of the user(s). Further, embodiments herein disclose a seat cover with integrated sanitizer fluid flow channels and sanitizer dispensing unit to dispense the sanitized fluid to the user space of the vehicle. Embodiments herein disclose the seat cover which is simple in construction and allows retrofitting of sanitizer fluid flow channels and sanitizer dispensing unit therein to effectively dispense the sanitizer fluid to user space of the vehicle. Furthermore, embodiments herein disclose the system intimates the hygiene status of vehicle to the passenger before starting the ride. Referring now to the drawings, and more particularly to FIGS. 1 through 5, where similar reference characters denote corresponding features consistently throughout the figures, there are shown embodiments.

For the purpose of this description and ease of understanding, sanitizing the user space in the vehicle is explained herein with reference to a shared mobility vehicle. When a passenger book the trip or confirmed the vehicle for the trip, the system (100) detects the location of users (previous user or current user) to identify containment zone or non-containment zone or airport zone and hospital zone based on inputs received from global positioning system (GPS). Further, the system (100) can be configured to track the user(s) past travel history based on the need. Thereafter, the system (100) estimates the cost of ride and level of sanitization required in the user space of the vehicle based on at least one ride waiting time, sanitization time and sanitization premium and accordingly, the system (100) informs the estimated cost of ride and level of sanitization to the passenger. Thereafter, the passenger accepts the estimation level of sanitization or selects level of sanitization required in vehicle. Based on the confirmation, the driver rides the vehicle to the passenger location and then driver gets out from the vehicle and then the system automation takes over to complete the sanitization of the user space in vehicle as per passenger's selected level of sanitation and also sanitization process progress and hygiene status is informed to passenger. Then the passenger boards the vehicle after receiving notification about the hygiene status of the vehicle.

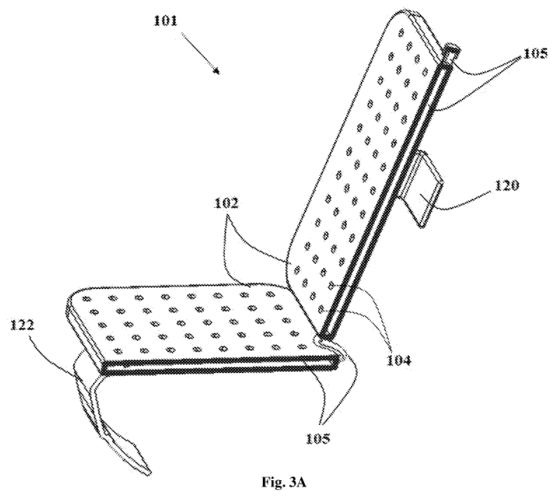

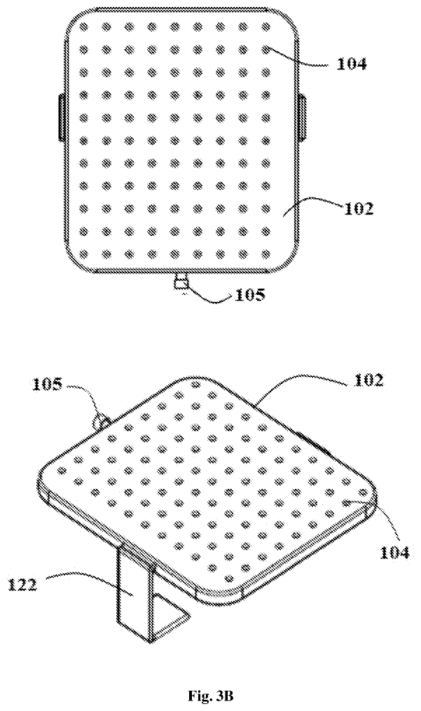

FIG. 1 depicts a sanitization system (100) in a vehicle, wherein the sanitization system (100) comprises a seat cover (101), sanitizing dispensing units (104), a sanitizer circulating device (106) and a controller unit (112), where the controller unit (112) is in communication with a sensor module (114) and an indication module (130), according to embodiments as disclosed herein. In the embodiment, the system (100) includes a module (102), a sanitizer storage tank (106), an auxiliary sanitizer storage tank (107), a sanitizer circulating device (110), a controller unit (112), a sensor module (114), at least one sanitizer flow conduit (118) and an indication module (130). In an embodiment, the module (102) includes a plurality of sanitizer dispensing units (104), a plurality of sanitizer fluid flow channels ((105), (as shown in FIG. 3a and FIG. 3b) and a cushioning jacket (not shown). The module (102) is integrated in one of seat or bed or seat cover or bed cover of the vehicle. For the purpose of this description and ease of understanding, the sanitization system (100) is explained herein below with reference to sanitizing the user space of vehicle by dispensing sanitizer fluid to the user space by one of retrofitting or integrating sanitizer dispensing units (104) and sanitizer fluid flow channels (105) in at least one seat cover (101) of the vehicle, where the system (100) effectively sanitizes the user space and maintain the hygiene conditions in the vehicle thereby protecting passengers from exposure to potentially harmful microbes on passenger seats as well as interior space in vehicle. However, it is also within the scope of the invention to use/implement the sanitization system (100) for sanitizing the user space and maintain the hygiene conditions and also to protect passengers from exposure to potentially harmful microbes on beds or ambulance bed covers, door handles, dashboard, roof, door, and heating, ventilation and air conditioning (HVAC) module or any other location in interior space of the vehicle or any other applications, where sanitizing and hygiene is required. The seat cover (101) is attached to the seats (124, 126) of the vehicle with two non-permanent attachments (120, 122) defined on the seat cover (101).

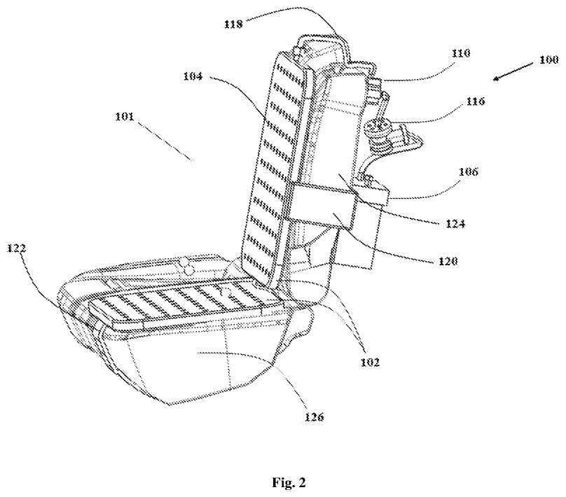

FIG. 2 depicts the seat cover (101) retrofitted onto a seat (124, 126) of the vehicle, according to embodiments as disclosed herein. The seat (124, 126) includes a seating portion (126) and a non-seating portion (124). The seat cover (101) includes non-permanent attachments (120, 122) adapted to attach the seat cover (101) to the seat (124, 126) of the vehicle. FIG. 3A and 3B depict the seat cover (101) with module (102), wherein the module (102) comprises the at least one sanitizing dispensing unit (104) and sanitizer fluid flow channels (105), according to embodiments as disclosed herein. The sanitizer dispensing units (104) are positioned in the entirety of the seat cover (101) in a predefined pattern or configuration. Each sanitizer dispensing unit (104) act as nozzle to atomize the sanitizer fluid and dispense the atomized sanitizer fluid into vehicle interior space thereby sanitizing the user space. The sanitizer fluid flow channel (105) of the module (102) is in fluid communication with the sanitizer dispensing units (104) and the sanitizer circulating device (110). The sanitizer fluid flow channel (105) is adapted to allow sanitizer fluid flow from the sanitizer circulating device (110) to the sanitizer dispensing units (104). The cushioning jacket of the module (102) is adapted to provide comfort to the user. The cushioning jacket is interposed between the sanitized fluid flow channel (105) or the sanitizer dispensing unit (104) and one of seat or bed or seat cover or bed cover. The cushioning jacket of the module (102) is considered to be one of a water jacket and a foam jacket. In one embodiment, the sanitizer dispensing unit (104) and the sanitizer fluid flow channel (105) is integrated as a single layer and is overlaid on the cushioning jacket. In another embodiment, the sanitizer dispensing unit (104), the sanitizer fluid flow channel (105) and the cushioning jacket is provided as an integrated single unit or as separate parts.

The sanitizer storage tank (106) is adapted to store sanitizer fluid. The auxiliary sanitizer storage tank (107) is adapted to store the unutilized sanitizer fluid which is re-circulated by the sanitizer circulating device (110) from the sanitized user space. The sanitizer circulating device (110) in fluid communication (118) with the sanitizer storage tank (106), the sanitizer dispensing unit (104) and the auxiliary sanitizer storage tank (107).

The controller unit (112) is in communication with the sanitizer circulating device (110), the sensor module (114) and the indication module (130). The controller unit (112) is adapted to initiate a sanitizing cycle in which the controller unit (112) activates the sanitizer circulating device (110) based on said at least one sensory information received from the sensor module (114), where the sensory information is a parameter relevant to sanitizing the user space of the vehicle. In the sanitizing cycle, the sanitizer circulating device (110 is configured to act as a compressor which is adapted to circulate sanitizer fluid from the sanitizer storage tank (106) to the at least one sanitizer dispensing unit (104) which in turn dispenses the sanitizer fluid across the seat cover (101) and the user space of vehicle when the sanitizer circulating device (110) receives instructions from the controller unit (112).

The controller unit (112) is adapted to initiate a sanitizer regeneration cycle in which the controller unit (112) activates the sanitizer circulating device (110) based on at least one sensory information received from the sensor module (114). In sanitizer regeneration cycle, the controller unit (112) reverses the direction of electric current supply to the sanitizer circulating device (110) thereby reversing the direction of rotation of the sanitizer circulating device (110) based on at least one sensory information received from the sensor module (114). In the sanitizer regeneration cycle, the sanitizer circulating device (110) is configured to act as a suction pump (vacuum pump) which is adapted to circulate unutilized sanitizer fluid from the sanitized user space to the auxiliary sanitizer storage tank (107) through the sanitizer dispensing unit (104), the sanitizer fluid flow channels (105) and the sanitizer flow conduit (118). The unutilized sanitizer fluid collected in the auxiliary sanitizer storage tank (107) can be re-directed to the sanitizer storage tank (106). Before reusing the unutilized sanitizer fluid, the system (100) checks the dilution levels of the unutilized sanitizer fluid in the auxiliary sanitizer storage tank (107). The system (100) reuses the unutilized sanitizer fluid for dispensing into the user space of a vehicle based on the dilution check of the unutilized sanitizer fluid in the auxiliary sanitizer storage tank (107).

The sensor module (114) is adapted to detect and communicate at least one sensory information to the controller unit (112). The sensory information sent by the sensor module (114) to the controller unit (112) is at least one of a parameter relevant to sanitizing the user space of the vehicle, and a parameter relevant to sanitizer regeneration from the sanitized user space. The sensor module (114) comprises various sensors which are arranged in vehicle user space i.e. in the cabin area, where the various sensors are GPS (Global positioning sensor), occupant present sensor, humidity sensor, sanitizer level monitoring sensor and camera. The sensory information sent by the sensor module (114) to the controller unit (112) to initiate the sanitizing cycle is at least one of geographical information, trip status, occupant presence and input(s) from the user(s). The sensory information sent by the sensor module (114) to the controller unit (112) to initiate sanitizer regeneration cycle is at least one of humidity level in the user space, duty cycle of sanitizer circulating device (110) and sanitizer level in the user space. The GPS (global positioning sensor) can measure the trip status and passenger location and communicate the measured information to the controller unit (112). In an embodiment herein, the GPS can communicate the measured information of the passenger location and trip status to the controller unit (112) using a wireless means. In an embodiment herein, the GPS can communicate the measured information of the passenger location and trip status to the controller unit (112) using a wired. The occupant present sensor is adapted to detect and communicate occupant presence status to the controller unit (112). The humidity sensor is adapted to detect and communicate the humidity level of sanitizer in the user space to the controller unit (112). The sanitizer level monitoring sensor is adapted to detect and communicate the sanitizer level in the user space to the controller unit (112). Further, the sensor module (114) includes a monitoring sensor which monitors the duty cycle or run time of the sanitizer circulating device (110). For the purpose of this description and ease of understanding, the monitoring sensor monitors the duty cycle or run time of the sanitizer circulating device (110) is considered to be a timer. The camera is adapted to capture and communicate at least one media input (image or video) of the user space captured the sanitizing process in the vehicle, to the controller unit (112). The controller unit (112) provides at least one signal to the indication module (130) based on the information received from the sensor module (112).

The first non-return valve (116) is adapted to allow sanitizer fluid flow from the sanitizer storage tank (106) to the sanitizer circulating device (110), where the first non-return valve (116) is adapted to restrict sanitizer return flow to the sanitizer storage tank (106). The second non-return valve (117) is adapted to allow ambient air flow to the sanitizer storage tank (106). The sanitizer flow conduit (118) is adapted to allow sanitizer fluid flow from the sanitizer circulating device (110) to the sanitizer fluid flow channel (105). One end of the sanitizer flow conduit (118) is connected to the sanitizer circulating device (110) and another end of the sanitizer flow conduit (118) is connected to the sanitizer fluid flow channel (105).

In the embodiment, the indication module (130) is adapted to indicate the sanitization status to the user, based on instruction received from the controller unit (112), wherein the indication module (130) is at least one of an audio indicating means, a visual indicating means, user interface unit and olfactory indicator. For the purpose of this description and easy understanding, the indication module (130) provides the indication signal to the car infotainment system or message alert to the user mobile phone or any electronic communication device of the passenger and/or driver. The indication module (130) provides indication via audio means or visual means i.e. voice information about the status of the hygiene in the vehicle or display alerts in the car infotainment system or personal electronic devices of the user(s).

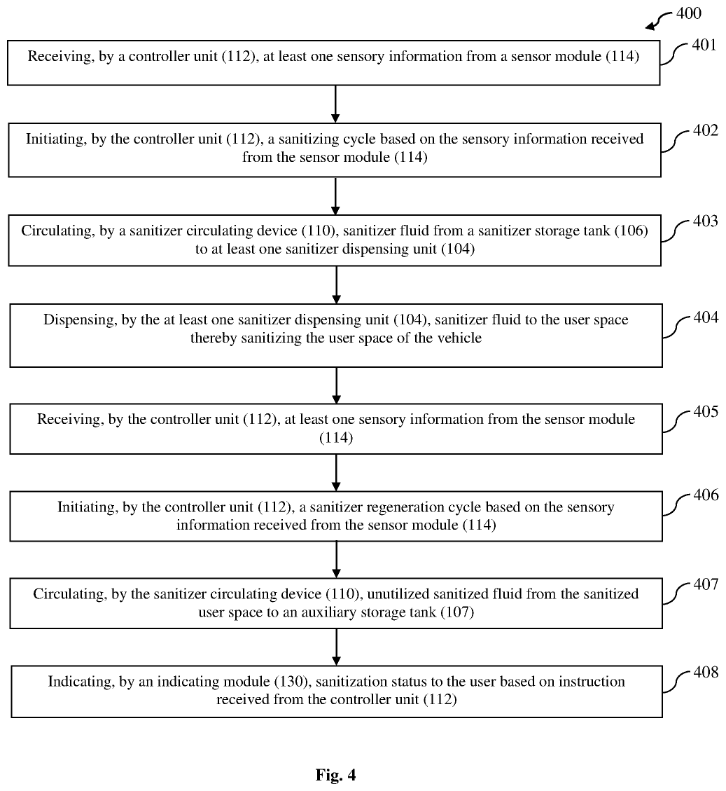

FIG. 4 is a flowchart depicting a process (400) of sanitizing the user space and regeneration of sanitizer fluid from the sanitized user space of the vehicle, according embodiments as disclosed herein. The various actions in method (400) may be performed in the order presented, in a different order or simultaneously. Further, in some embodiments, some actions listed in FIG. 4 may be omitted.

At step 401, the method (400) includes, receiving, by a controller unit (112), at least one sensory information from a sensor module (114). In an embodiment herein, the sensory information can be used to provide a trigger to the controller unit (112) to activate the sanitizer circulating device (110) manually. In another embodiment herein, the sensory information can be used to provide a trigger to the controller unit (112) to activate the sanitizer circulating device (110) automatically.

At step 402, the method (400) includes, initiating, by the controller unit (112), a sanitizing cycle based on the sensory information received from the sensor module (114), where the sensory information is a parameter relevant to sanitizing the user space of the vehicle.

At step 403, the method (400) includes, circulating (403), by the sanitizer circulating device (110), sanitizer fluid (108) from a sanitizer storage tank (106) to at least one sanitizer dispensing unit (104) when the sanitizer circulating device (110) is activated by the controller unit (112). In the sanitizing cycle, sanitizer circulating device (110) is configured to act as compressor. The controller unit (112) calculates the duty cycle or run time of the sanitizer circulating device (110) based on the sensory information from the sensor module (112).

At step 404, the method (400) includes dispensing, by the at least one sanitizer dispensing unit (104), the sanitizer fluid to the user space thereby sanitizing the user space. The sanitizer dispensing unit (104) acts as nozzle to dispense the atomized sanitized fluid at sufficient pressure thereby the dispensed sanitizer fluid dispensed covers the maximum area in the vehicle cabin including the seat area.

Once the user space is sanitized, at step 405, the method (400) includes, receiving, by the controller unit (112), at least one sensory information from the sensor module (114).

At step 406, the method (400) includes initiating (406), by the controller unit (112), a sanitizer regeneration cycle based on the sensory information received from the sensor module (114), where the sensory information is a parameter relevant to sanitizer regeneration from said user space. The sensory information includes humidity level and sanitization levels in the user space, and duty cycle of the sanitizer circulating device (110).

At step 407, the method (400) includes circulating (407), by the sanitizer circulating device (110), unutilized sanitized fluid from said sanitized user space to an auxiliary sanitizer storage tank (107) when said sanitizer circulating device (110) is activated by said controller unit (112. In an embodiment herein, after step 406 and prior to step 407, the method (400) includes reversing, by the controller unit (112), direction of rotation of the sanitizer circulating device (110) by changing the direction of current in the sanitizer circulating device (110) by using H-bridge circuit, by this reverse polarity is created thus the sanitizer circulating device (110) switches from acting as compressor to vacuum pump or suction pump to draw the unutilized sanitized fluid from the sanitized user space to the auxiliary sanitizer tank (107).

Further, the method (400) includes, indicating, by an indication module (130), sanitization status to the user, based on instruction received from the controller unit (112), wherein the indication module (130) is at least one of an audio indicating means, a visual indicating means, a user interface unit and an olfactory indicator.

Further, the method (400) includes providing at least one sanitizer flow channel (105) in fluid communication with the at least one sanitizer dispensing unit (104) of the module (102). Further, the method (400) includes connecting one end of a sanitizer flow conduit (118) to the sanitizer circulating device (110) and connecting another end of the sanitizer flow conduit (118) to the sanitizer flow channel (105). Further, the method (400) includes interposing at least one cushioning jacket between the at least one sanitizer dispensing unit (104) or the sanitizer flow channel (105) and one of the seat or bed or seat cover or bed cover. Furthermore, the method (400) includes allowing, by a first non-return valve (116), sanitizer fluid flow from said sanitizer storage tank (104) to said sanitizer circulating device (110). Further, the method (400) includes allowing, by a second non-return valve (117), ambient air flow to the sanitizer storage tank (107).

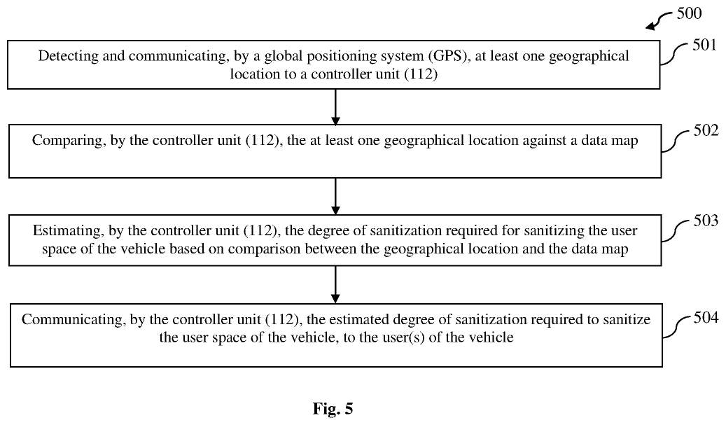

FIG. 5 is a flowchart depicting a process (500) of estimating the degree of sanitization level required in user space of the vehicle, according to embodiments as disclosed herein. The various actions in method (500) may be performed in the order presented, in a different order or simultaneously. Further, in some embodiments, some actions listed in FIG. 5 may be omitted.

Considering that before imitating/alerting the sanitization process to the users, the sanitizer advisory system estimates the degree or level of sanitization required to sanitize the user space of the vehicle. At step 501, the method (500) includes detecting and communicating, by a global positioning system (GPS), at least one geographical location to a controller unit (112).

At step 502, the method (500) includes, comparing (502), by the controller unit (112), the at least one geographical location against a data map, where the data map includes the history of non-containment zones, hospitals, airports and containment zones created as per the covid guide line by the state governments. In another embodiments, the data map pertains to the passenger pick up location having higher chance of spreading the virus or containment zones for example if the passenger is in the location of hospitals or near by isolation zones or airports or/and other risk areas. In those places the chance of spreading the virus is high. Further, the data map includes past travel history of user(s).

At step 503, the method (500) includes, estimating (503), by the controller unit (112), the degree of sanitization required for sanitizing the user space of the vehicle based on comparison between the at least one geographical location and the data map.

At step 504, the method (500) includes communicating (504), by the controller unit (112), the estimated degree of sanitization required to sanitize the user space of the vehicle, to user(s) of the vehicle.

The method step (503) of estimating (503), by the controller unit (112), the degree of sanitization required for sanitizing the user space of the vehicle based on comparison between the at least one geographical location and the data map, includes categorizing, by the controller unit (112), the estimated degree of sanitization into high, medium, and low level of sanitization required for sanitizing the user space of the vehicle, based on comparison between the at least one geographical location and the data map, prior to the method step (504) of communicating, by controller unit (112), the estimated degree of sanitization required to sanitize the user space of the vehicle, to the user of the vehicle.

The method step of categorizing, by the controller unit (112), the estimated degree of sanitization into high, medium, and low level of sanitization required for sanitizing the user space of the vehicle includes at least one of calculating by, the controller unit (112), a duty cycle of a sanitizer circulating device (110); and varying by, the controller unit (112), power supply to the sanitizer circulating device (110) for the categorized degree of sanitization required to sanitize the user space of the vehicle. The at least one geographical location detected and communicated by the GPS to the controller unit (112) is at least one of a start ride location of previous passenger, a stop ride location of the previous passenger, a current location of the vehicle before start of next ride and a location of current passenger intending to use the vehicle.

In another embodiments, categorizing, by the controller unit (112), the estimated degree of sanitization into rich, normal and lean levels of sanitization fluid required for sanitizing the user space of the vehicle based on the information received from the sensor module (112).

In further step, the controller unit (112) initiates the sanitizing cycle as per the instructions received from the users (passenger and driver). For ease of understanding, in an example as disclosed herein with respect to higher degree of sanitization required in user space of vehicle, the controller (112) communicates to the passenger whether user(s) (boarding passenger) needs high degree of sanitization required before staring the ride, where as in second scenario, if the passenger location is nearby any hospital areas or airports or isolation zone or containments, the controller unit (112) estimates the high level of sanitization, it means the controller unit (112) operates the sanitization circulating device (106) for high time period rather than normal time period, or by dispensing rich concentration of sanitizer fluid. In another embodiments herein, the controller unit (112) estimates degree of sanitization by operating the sanitizer circulating device (110) in one of a normal sanitization mode and a turbo sanitization mode.

The technical advantages of the system for sanitizing the user space of the vehicle are as follows. The system can effectively sanitize the user space in vehicle and intimating/alerting the hygiene status of vehicle before passenger starting the ride thereby protecting passengers from exposure to potentially harmful microbes accumulated on passenger seats as well other interior spaces in the vehicle. The seat cover which is simple in construction and allows retrofitting of sanitizer fluid flow channels and sanitizer dispensing unit therein to effectively dispense the sanitizer fluid to user space of the vehicle. The system estimates the degree of sanitization required in the user space of the vehicle in accordance to geographical location of the user(s). The system allows the user of vehicle to select the level of sanitization required in user space before starting the ride.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of embodiments and examples, those skilled in the art will recognize that the embodiments and examples disclosed herein can be practiced with modification within the spirit and scope of the embodiments as described herein.

We claim:

1. A system (100) for sanitizing user space of a vehicle, said system (100) comprising:
a module (102), said module (102) includes at least one sanitizer dispensing unit (104);
a sanitizer storage tank (106) adapted to store sanitizer fluid (108);
a sanitizer circulating device (110) in fluid communication with said sanitizer storage tank (106) and said sanitizer dispensing unit (104);
a controller unit (112) in communication with said sanitizer circulating device (110); and
a sensor module (114) adapted to sense and communicate at least one sensory information to said controller unit (112), wherein
said controller unit (112) adapted to initiate a sanitizing cycle in which said controller unit (112) activates said sanitizer circulating device (110) based on said at least one sensory information received from said sensor module (114), where said sensory information is a parameter relevant to sanitizing said user space of said vehicle;
said sanitizer circulating device (110) is adapted to circulate sanitizer fluid from said sanitizer storage tank (106) to said at least one sanitizer dispensing unit (104); and
said sanitizer dispensing unit (104) is adapted to dispense sanitizer fluid to said user space thereby sanitizing said user space.

2. The system (100) as claimed in claim 1, wherein said controller unit (112) is adapted to initiate a sanitizer regeneration cycle in which said controller unit (112) activates said sanitizer circulating device (110) based on at least one sensory information received from said sensor module (114), where said sensory information is a parameter relevant to sanitizer regeneration from said sanitized user space; and
said sanitizer circulating device (110) is to adapted to circulate unutilized sanitizer fluid from said sanitized user space to an auxiliary sanitizer storage tank (107) through said sanitizer dispensing unit (104).

3. The system (100) as claimed in claim 1, wherein said sensory information sent by said sensor module (114) to said controller unit (112) to initiate said sanitizing cycle is at least one of geographical information, trip status, occupant presence and input(s) from the user(s); and
said sanitizer circulating device (110) is configured to act as a compressor in said sanitizing cycle.

4. The system (100) as claimed in claim 2, wherein said sensory information sent by said sensor module (114) to said controller unit (112) to initiate sanitizer regeneration cycle is at least one of humidity level in said user space and sanitizer level in said user space, and duty cycle time of said sanitizer circulating device (110); and
said sanitizer circulating device (110) is configured to act as a suction pump (vacuum pump) in said sanitizer regeneration cycle.

5. The system (100) as claimed in claim 4, wherein said system (100) comprises,
a first non-return valve (116) adapted to allow sanitizer fluid flow from said sanitizer storage tank (106) to said sanitizer circulating device (110), where said first non-return valve (116) is adapted to restrict sanitizer return flow to said sanitizer storage tank (106);
a second non-return valve (117) adapted to allow ambient air flow to said sanitizer storage tank (106); and
a sanitizer flow conduit (118), one end of said sanitizer flow conduit (118) is connected to said sanitizer circulating device (110) and another end of said sanitizer flow conduit (118) is in fluid communication with sanitizer dispensing unit (104).

6. The system as claimed in claim 5, wherein said module (102) comprises at least one sanitizer fluid flow channel (105) in fluid communication with said sanitizer dispensing unit (104) and said sanitizer circulating device (110), wherein
said module (102) is integrated in one of seat, bed, seat cover and bed cover of said vehicle; and
said another end of said sanitizer flow conduit (118) is in fluid communication with said sanitizer dispensing unit (104) through said sanitizer fluid flow channel (105).

7. The system (100) as claimed in claim 6, wherein said module (102) comprises at least one cushioning jacket interposed between said sanitized fluid flow channel (105) or said at least sanitizer dispensing unit (104) and one of said seat, bed, seat cover and bed cover.

8. The system (100) as claimed in claim 1, wherein said system (100) comprises an indication module (130) adapted to indicate sanitization status to the user(s), based on instruction received from said controller unit (112), wherein said indication module (130) is at least one of an audio indicating means, a visual indicating means, a user interface unit and an olfactory indicator.

9. The system (100) as claimed in claim 7, wherein said at least one sanitizer dispensing unit (104) and said at least one sanitizer fluid flow channel (105) is integrated as a single layer and is overlaid on said cushioning jacket.

10. The system (100) as claimed in claim 7, wherein said at least one sanitizer dispensing unit (104), said sanitizer fluid flow channel (105) and said cushioning jacket is provided as an integrated single unit or as separate parts;
said sanitizer dispensing unit (104) is a nozzle; and
said cushioning jacket is one of a water jacket and a foam jacket.

11. A method (400) for sanitizing user space of a vehicle, said method (400) comprising:
receiving (401), by a controller unit (112), at least one sensory information from a sensor module (114);
initiating (402), by the controller unit (112), a sanitizing cycle based on the sensory information received from the sensor module (114), where the sensory information is a parameter relevant to sanitizing the user space of said vehicle;
circulating (403), by the sanitizer circulating device (110), sanitizer fluid (108) from a sanitizer storage tank (106) to at least one sanitizer dispensing unit (104) when said sanitizer circulating device (110) is activated by said controller unit (112); and
dispensing (404), by the at least one sanitizer dispensing unit (104), the sanitizer fluid (108) to said user space thereby sanitizing said user space.

12. The method (400) as claimed in claim 11, wherein the sensory information sent by said sensor module (114) to said controller unit (112) to initiate said sanitizing cycle is at least one of geographical location(s) of user(s), trip status, occupant present status and input(s) from the user; and
said sanitizer circulating device (110) is configured to act as a compressor in the sanitizing cycle.

13. The method (400) as claimed in claim 11, wherein said method (400) includes,
receiving (405), by the controller unit (112), at least one sensory information from the sensor module (114);
initiating (406), by the controller unit (112), a sanitizer regeneration cycle based on the sensory information received from the sensor module (114), where the sensory information is a parameter relevant to sanitizer regeneration from said user space;
reversing, by the controller unit (112), direction of rotation of the sanitizer circulating device (110) by changing the direction of current in the sanitizer circulating device (110); and
circulating (407), by the sanitizer circulating device (110), unutilized sanitized fluid from said sanitized user space to an auxiliary sanitizer storage tank (107) when said sanitizer circulating device (110) is activated by said controller unit (112), wherein
said sensory information sent by said sensor module (114) to said controller unit to initiate said sanitizer regeneration cycle is at least one of humidity level and sanitizer level in said user space and duty cycle time of said sanitizer circulating device (110); and
said sanitizer circulating device (110) is configure to act as a suction pump (vacuum pump) in said sanitizer regeneration cycle.

14. The method as claimed in claim 11, wherein said method (400) comprises indicating, by an indication module (130), sanitization status to the user(s), based on instruction received from said controller unit (112), wherein said indication module (130) is at least one of an audio indicating means, a visual indicating means, a user interface unit and an olfactory indicator.

15. The method (400) as claimed in claim 11, wherein said method (400) includes,
providing at least one sanitizer fluid flow channel (105) in fluid communication with said at least one sanitizer dispensing unit (104) of the module (102);
connecting one end of a sanitizer flow conduit (118) to said sanitizer circulating device (110) and connecting another end of said sanitizer flow conduit (118) to said sanitizer flow channel (105);
interposing at least one cushioning jacket between said sanitizer dispensing unit (104) or said sanitizer flow channel (105) and one of said seat, bed, seat cover and bed cover;
allowing, by a first non-return valve (116), sanitizer fluid flow from said sanitizer storage tank (104) to said sanitizer circulating device (110); and
allowing, by a second non-return valve (117), ambient air flow to said sanitizer storage tank (107), wherein
said first non-return valve (116) is adapted to restrict sanitizer return flow to said sanitizer storage tank (104);
said at least one sanitizer dispensing unit (104), sanitizer flow channel (105) and said cushioning jacket is provided as separate parts or as an integrated single unit in seat cover or bed cover; and
said sanitizer dispensing unit (104) is a nozzle; and
said cushioning jacket is one of a water jacket and a foam jacket.

16. A method (500) for estimating degree of sanitization required in user space of a vehicle, said method (500) comprising:
detecting and communicating (501), by a global positioning system (GPS), at least one geographical location to a controller unit (112);
comparing (502), by said controller unit (112), said at least one geographical location against a data map;
estimating (503), by said controller unit (112), the degree of sanitization required for sanitizing said user space of said vehicle based on comparison between said at least one geographical location and said data map; and
communicating (504), by said controller unit (112), said estimated degree of sanitization required to sanitize said user space of said vehicle, to user(s) of said vehicle.

17. The method (500) as claimed in claim 16, wherein said estimating (503), by said controller unit (112), the degree of sanitization required for sanitizing said user space of said vehicle includes,
categorizing, by said controller unit (112), said estimated degree of sanitization into high, medium, and low level of sanitization required for sanitizing said user space of said vehicle, based on comparison between said at least one geographical location and said data map, prior to said communicating (504), by said controller unit (112), said estimated degree of sanitization required to sanitize said user space of said vehicle to the user of said vehicle, wherein
said categorizing, by said controller unit (112), said estimated degree of sanitization into high, medium, and low level of sanitization required for sanitizing said user space of said vehicle includes at least one of, calculating by, said controller unit (112), a duty cycle of a sanitizer circulating device (110); and varying by, said controller unit (112), power supply to said sanitizer circulating device (110) for the categorized degree of sanitization required to sanitize said user space of said vehicle;
the at least one geographical location detected and communicated by said GPS to said controller unit (112) is at least one of a start ride location of previous passenger, a stop ride location of the previous passenger, a current location of said vehicle before start of next ride and a location of current passenger intending to use said vehicle; and
said data map includes history of containment zones, non-containment zones, hospitals, airports and past travel history of user(s).
